(19) United States
(12) Patent Application Publication
(10) Pub. No.: US 2021/0290785 A1
(43) Pub. Date: Sep. 23, 2021

(54) METHOD OF FORMING AND SEPARATING TIN(II) FROM TIN(IV)

(71) Applicant: SnIP Holdings, Incorporated, The Woodlands, TX (US)

(72) Inventors: Druce Crump, Lake Jackson, TX (US); Jaime Simon, Angleton, TX (US); Nigel R. Stevenson, Sugar Hill, GA (US)

(73) Assignees: SnIP Holdings, Incorporated, The Woodlands, TX (US); SnIP Holdings, Incorporated, The Woodlands, TX (US)

(21) Appl. No.: 16/338,754

(22) PCT Filed: Oct. 5, 2017
(86) PCT No.: PCT/US2017/055246

§ 371 Date: Apr. 2, 2019

Related U.S. Application Data
(60) Provisional application No. 62/404,892, filed on Oct. 6, 2016.

(51) Int. Cl. A61K 51/04; C07F 7/22; A61K 47/54; C07B 59/00

(52) U.S. Cl. CPC A61K 51/0482; C07F 7/2284; C07B 2200/05; C07B 59/004; A61K 47/547

(57) ABSTRACT

Tin(IV)-117m is reduced by an acidic antimony solution to tin(II)-117m. A chelant is added to the solution of tin(II)-117m, and the pH is raised, forming tin(II)-117m chelant complex. The chelant is a bifunctional chelant, preferably attached to a bioactive molecule and reacted with the tin(II)-117m to form a radiopharmaceutical.

### BACKGROUND OF THE INVENTION

Radioactive tin-117m can be used as a radiopharmaceutical. Highly purified no-carrier-added tin-117m is preferred. Typical methods of producing tin-117m and, in particular, no-carrier-added tin-117m, generally produce the tin-117m in the +4 state.

In most applications, to be used as a radiopharmaceutical, the tin-117m must be attached to a bioactive molecule. The bioactive molecule can perform several functions, such as directing the tin-117m to a desired location. Generally, the tin(IV) is chelated to a molecule that has four charge centers, such as carboxylic acid groups or amine groups, that accept the tin(IV). The reaction, however, takes place under relatively severe conditions which can affect the yield.

### SUMMARY OF THE INVENTION

The present invention is premised on the realization that tin(IV)-117m can be reduced to tin(II)-117m, conjugated to a bifunctional chelating agent attached to a bioactive molecule, and then subsequently reacted to form tin(IV), which is strongly bonded to the chelating agent.

### DETAILED DESCRIPTION

According to the present invention, tin(II) is bonded to a chelating agent and allowed to react and form tin(IV) while bonded to the chelating agent. In one embodiment, tin(IV)-117m is first reduced by an acidic solution of, for example, antimony to form tin(II). The tin(II), in turn, can be reacted with a bifunctional chelating agent, such as aminobenzyl DOTA, which can then be easily separated from the acidic antimony solution. Preferably, the chelating agent will be bonded to an ioactive molecule prior to being reacted with the tin(II). According to the present invention, the tin(IV)-117m is heated in the presence of antimony at low pH. The bifunctional chelating agent is then added to the acidic solution and the pH raised to about 5 to 6. The reduced tin(II) attaches to the chelating agent under very mild conditions and the tin(II) will subsequently naturally convert to tin(IV) without the addition of an oxidizing agent.

For use in the present invention, the tin-117m in the +4 state can be obtained according to any well-known methods. The tin can be carrier added or no-carrier added, depending on its intended use. One preferred method of forming high specific activity no-carrier-added tin-117m is disclosed in U.S. Pat. No. 8,257,681, the disclosure of which is hereby incorporated by reference. This produces an extremely pure no-carrier-added tin-117m in the +4 state.

Any bifunctional chelant which will bind to the tin(II) will function. Generally, these will be chelants having four charge centers, such as carboxylic acid or amine groups. Any chelant can be used in the present invention, so long as the chelating agent can bond to a second molecule, such as, for example, annexin. The second molecule or bioactive molecule is one which directs the tin-117m to an appropriate location in the body, or has other functions within the body, which facilitates treatment or imaging of an ailment in the human body.

One such chelating agent is aminobenzyl-DOTA. The DOTA portion of the molecule has four active carboxylic acid groups and is known to chelate with tin(IV). The aminobenzyl portion of the molecule that can react with other biomolecules, for example annexin.

### Procedure for Reducing Tin(IV) Using Sb

According to the following procedure, one molar solution of hydrochloric acid is prepared and combined with 0.1 molar SnCl4.5H₂O. About 200 μL of 0.1 molar Sn(IV) solution is combined with an excess of antimony, about 25 mg. This is heated at 50° C. for thirty minutes in water bath. Supernatant tin solution is combined with 0.1 molar aminobenzyl-DOTA solution 1:1. PH is adjusted to five, using four molar NaOAc or, alternately, the pH can be raised to six using sufficient one molar MES buffer solution. Tin(II) aminobenzyl-DOTA remains in solution while the antimony precipitates out and can be separated by filtration. This allows the tin(II) chelate to be formed and separated from the reducing reagents. HPLC confirmed the reaction of the tin(II) to the aminobenzyl-DOTA.

The tin(II) aminobenzyl-DOTA then naturally oxidizes (for example in the presence of oxygen) to tin(IV) aminobenzyl-DOTA, which is a much more stable compound.

The present invention enables one to form the Sn(II)-117m and ship this to a radiopharmacy, where it can be reacted with, for example, aminobenzyl-DOTA annexin or other chelant bioactive molecule. This will then form Sn(IV)-117m and can be injected into a patient. This invention also lends itself to distribution using a kit. A kit will include the tin(IV)-117m in one vial. The components necessary for the antimony reduction would be in a second vial. The radiopharmacy may have the aminobenzyl-DOTA—bioactive molecule on hand or it can be shipped in a third vial. The tin(II) is formed by combining the tin(IV) and the antimony reduction solution. The tin(II) is then added to the chelant bioactive molecule solution and reacted. The tin-117m will naturally oxidize to tin(IV) 117m and is ready for injection.

This has been a description the present invention and the preferred method of practice in the present invention. However, the invention itself should only be defined by the appended claims wherein we claim:

What is claimed is:

1. A method of forming a tin chelant comprising:
reducing tin(IV) in the presence of antimony under acidic conditions to form a solution of tin(II);
adding a chelating agent to said solution of tin(II) and raising the pH of said solution, thereby causing the tin(II) to bind to the chelating agent and causing any residual antimony to precipitate from the solution.

2. The method claimed in claim 1, wherein said tin(IV) is tin-117m.

3. The method claimed in claim 1 wherein said chelant is bound to a bioactive molecule.

4. The method claimed in claim 1, wherein said tin(II) chelant is allowed to oxidize to tin(IV).

5. The method claimed in claim 1, wherein said chelant is a bifunctional chelant.

6. The method claimed in claim 5, wherein said chelant has four charge centers.

7. The method claimed in claim 6, wherein said chelant is aminobenzyl-DOTA.

8. A kit comprising:
a first solution, including tin(IV) 117 m, and
a second solution comprising an acidic solution of antimony effective to reduce said tin(IV) 117m to tin(II) 117m.

9. The kit claimed in claim 8 further comprising a chelant for said tin(II) 117m bonded to a bioactive molecule.

10. A method of forming a tin chelant comprising bonding tin(II) to a chelating agent and allowing said tin(II) to form tin(IV) while attached to said chelating agent.
